(19) United States
(12) Patent Application Publication
(10) Pub. No.: US 2006/0246564 A1
(43) Pub. Date: Nov. 2, 2006

(54) Immobilized biological material with improved functionality and method for producing the same

(76) Inventors: Carmen Parent, Quebec (CA); Anne Belzil, Levis (CA); Michelle Plante, Sainte-Foy (CA); Richard Lacasse, Beauport (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/411,774

(22) Filed: Apr. 27, 2006

Related U.S. Application Data
(60) Provisional application No. 60/675,484, filed on Apr. 28, 2005.
(60) Provisional application No. 60/702,632, filed on Jul. 27, 2005.

(51) Int. Cl. C12N 11/06

(52) U.S. Cl. 435/181

(57) ABSTRACT

The present invention provides methods and systems for performing biological reactions with biologically active entities immobilized on a solid support. Particularly, the invention provides a biologically active entity immobilized a support through a spacer linked to a first linker. The spacer gives the biologically active entity a certain level of free movement relatively to the support surface to which it is fixed, thereby allowing the biological reaction to take place.

### CROSS-REFERENCE TO RELATED APPLICATIONS

This application claims priority on U.S. provisional applications Ser. No. 60/675,484 filed Apr. 28, 2005, and Ser. No. 60/702,632 filed Jul. 27, 2005, the entire content of which are hereby incorporated by reference.

### FIELD OF THE INVENTION

The present invention concerns the field of methods and systems for immobilizing biologically active entities, such as enzymes, and performing biological reactions with the immobilized biologically active entities. The present invention also concerns methods and systems for converting CO₂ into environmental-friendly by-products.

### BACKGROUND OF THE INVENTION

The increasing presence of carbon dioxide in the environment deserves particular attention because it contributes to the greenhouse effect. Presently, the annual quantity of carbon that is emitted in the form of CO₂ is evaluated to be 7.2 billion tons, of which 5.5 billion tons result from the combustion of fossil fuels and 1.7 billion tons result from deforestation mainly in tropical zones. Although a large part of the carbon dioxide is neutralized by the oceans and the plants that are found in the Earth's forests, 1.9 billion tons of this gas per year remains non converted and non treated, and thus accumulated in the atmosphere.

Different methods for the recovery or the conversion of CO₂ into carbonate and hydrogen have been developed recently. In addition, different methods and systems limiting the emission of gaseous carbon dioxide have also been developed. Many systems resorting to microorganisms or chemical reactions for the recovery, treatment and conversion of carbon dioxide have been described in several documents, such as in U.S. Pat. No. 3,853,712, U.S. Pat. No. 4,047,894, U.S. Pat. No. 5,061,455 and U.S. Pat. No. 5,972,684.

However, none of these technologies has relied on the use of biological systems, such as systems comprising enzymes, for the degradation or conversion of CO₂ into reusable by-products. In particular, carbonic anhydrase is described in U.S. Pat. No. 6,524,843 which mentions the use of this enzyme in the system of gas extraction comprising carbon dioxide. Carbonic anhydrase, also called dehydratase carbonate, catalyses the hydration of carbon dioxide.

The first researches dealing with carbonic anhydrase have shown that the enzyme plays a crucial role in maintaining the pH of bodily fluids. It is known that a large quantity of acid substances are generated in the human or animal body, through the dietary or metabolic routes and interfere with intra and extracellular media. Eight (8) forms of carbonic anhydrase have been described in the prior art to this day. They are present, in particular, in mammals and in plants.

Carbonic anhydrase was used in many studies for the development of methods dealing with protein fixations on different supports or in various systems used for exploiting the characteristics of these proteins. The literature is replete with techniques for the immobilization of enzymes on different solid supports for different uses in reactors. Each enzyme requires specific conditions of the micro-environment to be able to maintain an acceptable catalytic capacity, but also to be able to be positioned so that the active site of the enzyme can meet its substrate. During an immobilization, many factors must be considered and are susceptible of significantly influencing the immobilization yield as well as the activity of the enzyme.

Immobilization of biological material on a solid support was widely studied and is widely used in many fields of activity. Enzymes alone or cell components (bacteria, yeasts, etc) or even whole cells, are biologically active entities which, in certain cases, permit the catalysis of a large number of biochemical and biological reactions, whether they are used for the transformation of foods or the treatment of liquid or gaseous effluents. Many immobilization methods have been developed by involving covalent, ionic bonds, or more simply through a phenomenon of adsorption, for fixing biologically active entities. When these methods have to be used on an industrial scale, it is important that they meet certain criteria in order to facilitate their application to a maximum degree.

Many methods of immobilization using covalent bonds involving “spacer” molecules and binding molecules have been developed in the last ten years. Some reactors that use a support that is immobilized with biological material have multiple configurations such as that of lined column, fluid bed or still membrane reactor. For example, Isgrove et al. (Enz. Microbiol Tech., 2001, 28: 225-232) describe a method of immobilizing β-glucosidase and trypsin on nylon supports.

Immobilizing enzymes on a solid support is carried out through a series of steps and washings which makes the technique not only time consuming but also costly. Many variants depending on the active biologically entity that is used, have been developed.

Immobilization of enzymes, i.e. their fixation on a solid support, such as polymers, has constituted an important progress in this technical field. It has brought a considerable improvement, in that it has made it possible to re-use an enzyme many times which, otherwise, in free state, was lost or destroyed between operations. In addition, working with immobilized enzymes contributes to the purity of the treated medium. However, fixation of the enzymes on solid supports, whether it is carried out by adsorption or through covalent bonds, has, to this day, remained an operation which is quite delicate, and which requires a well documented choice of materials and conditions. Immobilization through covalent bonds leads to more stable systems, avoiding losses of enzymes during their utilization. However, the enzymatic system obtained by this last mentioned method, generally presents a lower activity than the free enzyme since its immobilization leads to physical constraints. As a matter of fact fixation of a plurality of proteins on a rigid support can reduce all accessibility of the substrate to the active sites of the macromolecule. It is known that the activity of ribonuclease, when it is immobilized on agarose, is reduced in the order of 10 to 75% as compared to the free enzyme, depending on the nature of the treated substrate. To overcome this disadvantage, it is possible to use an additional intermediate bond, i.e. an intermediate reactant, combined on the one hand with the support and on the other hand with the enzyme. However, even with known systems of this type, the enzymatic activity is generally lower than that of the free enzyme. On the other hand, the choice of agents that can play the role of additional intermediate bond is somewhat restricted, since many possible reactants modify the activity of the enzyme, or else the preparatory conditions that are involved, are not compatible with preserving catalytic activity.

In view of the art described above, few methods and systems allow for the fixation of an active biologically entity while preserving its activity to a maximum. Thus, new developments always remain in demand for this purpose. These systems could be applied to various fields, such as spatial exploration, industrial combustion of waste, transportation, etc.

### SUMMARY OF THE INVENTION

In a first aspect, the present invention provides improvements of the techniques for immobilizing a biologically entity on a solid support not only leading to an increase of the performance but also to a less costly implementation of the process and a largely simplified production.

In another aspect, the present invention provides means for the management of gaseous discharges, more particularly CO₂. Inter alia, the present invention concerns methods for immobilizing molecules capable of converting carbon dioxide on a support.

In yet another aspect, the present invention provides the immobilization of carbonic anhydrase on a support. In another aspect, the invention also provides an immobilized carbonic anhydrase that maintain catalytic carbon dioxide hydration activity.

In a further aspect, the present invention provides the possibility of modulating the chemistry of the support so as to modify the micro-environment of the immobilized enzyme. The new physico-chemical characteristics conferred to the support should take into consideration the catalysis requirements of the substrate and of the enzymatic active site.

In an aspect, the present invention provides a system for performing a biological reaction. The system consists essentially of (i) a support, (ii) a first spacer having a polyamine molecule, (iii) a first linker having a having a first aldehyde group and a second aldehyde group and (iv) a biologically active entity. In this particular system, the support is linked to the polyamine molecule of the spacer, the spacer is linked to the first polyaldehyde group of the first linker and the biologically active entity is linked to the second aldehyde group the first linker. In an embodiment, the system also contains a second linker having a first aldehyde group and a second aldehyde group. The first aldehyde group of the second spacer is linked to the polyamine molecule of the spacer and the second aldehyde group of the second linker is linked to said support. In an embodiment, the support is made of a compound selected from the group consisting of plastic, biopolymer, polytetrafluoroethylene (PTFE), ceramic, polyethylene, polypropylene, polystyrene, nylon, silica, carbonate, a derivative thereof and a combination thereof. In a further embodiment, the polyamine molecule of the spacer is selected from the group consisting of an hydrocarbon, an acyclic hydrocarbon an alkene, a polyene, a polyethylene, an imine and a polyethylenimine. In yet a further embodiment, the polyamine molecule of the spacer is hydrophilic and, in yet another embodiment, the polyamine molecule of the spacer is polyethylenimine. In yet another embodiment, the first linker and/or the second linker is (are) selected from the group consisting of glutaraldehyde, glutardialdehyde, 1,3-diformylpropane, glutaral, 1,5-pentanedial, 1,5-pentanedione and cidex and, in yet a further embodiment, the first linker and/or the second linker is (are) glutaraldehyde. In another embodiment, the biologically active molecule is an enzyme and, in a further embodiment, the enzyme is carbonic anydrase. In an embodiment, the biological reaction catalyzed by the system is the conversion of a toxic gaseous effluent into a lesser toxic by-product. In an embodiment, the biological reaction is the conversion of carbon dioxide into carbonate. In an embodiment, the biological reaction takes place is in aqueous solution.

In another aspect, the present invention provides a method for obtaining an immobilized biologically active entity. The method consists essentially in (i) providing a support linked to a spacer, the spacer having a polyamine molecule and being linked to a first linker, the first linker having a first aldehyde and a second aldehyde group, the first aldehyde group of the first linker being linked to the spacer, and (ii) linking a biologically active entity to the second aldehyde group of the first linker, thereby obtaining the immobilized biologically active entity. In an embodiment, the support is linked to a second linker, the second linker has a first aldehyde group and a second aldehyde group, the first aldehyde group of the second linker is linked to said support and the second aldehyde group of the second linker is linked to the spacer. Various embodiments of the support have been described above. In another embodiment, the support is hydrolyzed by acid or functionalized by an ammoniacal plasma treatment prior to step (i). In a further embodiment, the acid hydrolysis of the support causes the linking of the polyamine molecule of the spacer to the support. In a further embodiment, the acid hydrolysis of the support creates primary amine groups on the support. In yet a further embodiment, the primary amine group is selected from the group consisting of aminobutane, 2-amino-2-methylpropane, 1-methylaminopropane, dimethylpropane, sulfonamide, alkoxide, amide and Barton's base. Various embodiments of the polyamine molecule of the spacer have been described above. Various embodiments of the first linker and the second linker have been described above. Various embodiments of the biologically active molecule have been described above. In another embodiment, the steps of the method are performed at room temperature.

### BRIEF DESCRIPTION OF THE DRAWINGS

FIG. 1 illustrates the support used in Example I according to one embodiment of the present invention. The solid support (1) possesses primary amino groups (2) that are linked to a second linker comprising a polyaldehyde compound (3). The second linker is linked to a spacer having a polyamine molecule (4) being linked to a first linker having a polyaldehyde compound (3). The polyaldehyde compound of the first linker is linked to the biologically active entity (5).

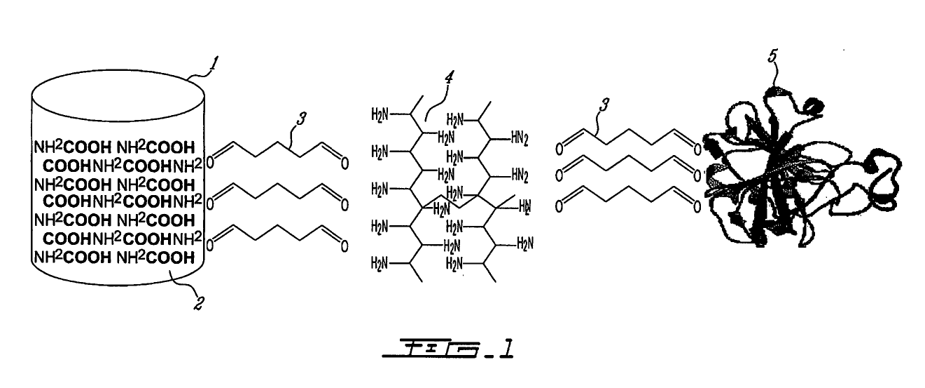

FIG. 2 illustrates the support used in Example II according to another embodiment of the invention. The solid support (1) is linked to a spacer having a polyamine molecule (4) being linker to a first linker having a polyaldehyde compound (3). The polyaldehyde compound of the first linker is linked to the biologically active entity (5).

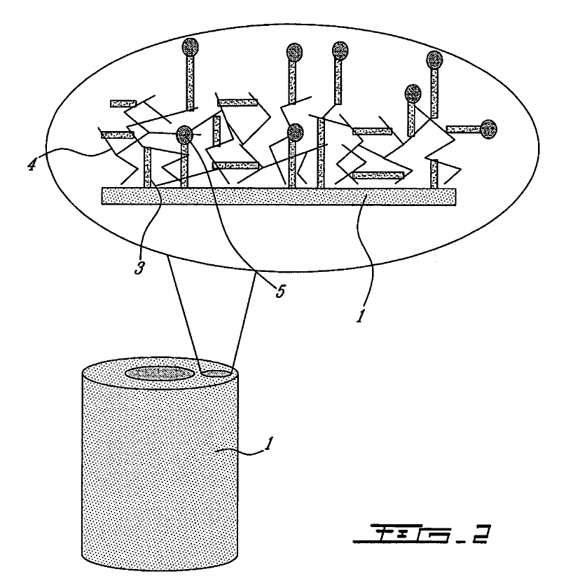

### DETAILED DESCRIPTION OF THE PREFERRED EMBODIMENTS

Immobilization of a biologically active entity on a relatively solid support was developed in order to allow for a covalent bond between the biologically active entity and a support while preserving its activity in an optimum manner. A method of immobilization of an biologically active entity on solid supports through covalent bonds, was thus developed.

According to one of the embodiments of the present invention, there is provided a system and method of producing it for immobilizing a biologically active entity on a support. As an example, but without limitation, this system may be used in large scale production in order to minimize production and operation costs, while preserving or increasing the rate of activity of the biologically active entity.

The system presented herein comprises a support, a spacer, a first linker and a biologically active entity. The support is directly or indirectly linked to the spacer. As used herein, the term “linked” is intended to mean the action of connecting two distinct entities. The link may be direct between the two entities, or it may be realized indirectly by means of another entity.

As used herein, the term “support” is intended to mean a solid or semi-solid material that immobilizes the biologically active entity. The support should interfere as little as possible with the biological reaction (such as the enzymatic reaction) that is being processed by the immobilized biologically active entity (such as an enzyme). The support should thus permit the activity of the biologically active entity. Various types of supports can be used in connection with the present invention. The supports may also include further components such as polyamides, polypropylenes (treated or functionalized with ammoniacal plasma), polystyrenes, etc. The supports may be configured for to meet their intended purpose. Such support include various geometries such as, but are not limited to Raschig™ rings, Tellerettes™, Tripack™, Pallring™, wires, membranes, etc.

The spacer can be fixed directly to the support without any specific treatment, or alternatively, the support's surface can be functionalized with primary amino groups to facilitate the linking of the first spacer. This can be done by treating the support's surface, depending on the material in which the support is made, to cause the creation of amine groups. The spacer can then be fixed to the support through the amine groups formed on the support surface. The support include, but is not limited to ceramic, polyethylene, polypropylene, polystyrene, nylon, silica, carbonate, a derivative thereof and a combination thereof. The support may also be enriched by the direct addition of amino groups, etc.

The type of support functionalization depends on the nature physico-chemical nature of the polymer that constitutes the support. For example, with respect to nylon-based support, functionalization is obtained by hydrolysis which is used to cut the chemical bonds of nylon and to produce amino and carboxyl groups. Depending on the type of support used, other treatments, such as acid or ammoniacal plasma treatments, can be used to functionalize the suppor'st surface. These treatments may generate primary amine groups on the surface of the support, thereby facilitating the linking of the spacer to the support.

The spacer of the system described herein may comprise a polyamine molecule. As used herein, the term “polyamine” is intended to mean a molecule having more than one amino group (or amine). Amines are organic compounds and a type of functional group that contain nitrogen as the key atom. Structurally amines resemble ammonia, wherein one or more hydrogen atoms are replaced by organic substituents such as alkyl and aryl groups. An important exception to this rule is that compounds of the type RC(O)NR₂, where the C(O) refers to a carbonyl group, are called amides rather than amines. Amides and amines have different structures and properties, so the distinction is chemically important. Somewhat confusing is the fact that amines wherein an N—H group has been replaced by an N-M group (M = metal) are also called amides. A polyamine molecule may contain amino groups (or amines) that can be found in simple linear or branched structure or still as grafted on an aromatic structure. The polyamine can have primary, secondary or tertiary amine groups or domains. The polyamine molecule can be for example, but without limitation, a polyethylenimine (PEI), chitosan, or any other hydrophilic molecule containing a plurality of amino groups. The hinge molecule is generally fixed to the polyamine according to standard techniques known in the art. The spacer may bind directly or indirectly, through its polyamine molecule, to the support described herein. Without wishing to be bound to any specific theory, the polyamine molecule of the first spacer is linked to the support's surface through adsorption forces, electrostatic forces, Van Der Waals forces, ionic bonds or hydrogen bonds. The spacer may also bind, indirectly, to the biologically active entity.

In an embodiment, the spacer provided in the system may be hydrophilic. Without wishing to be bound by theory, this physico-chemical property enables the spacer to facilitate the linking of the biologically active entity to the support.

Without wishing to be bound to theory, the spacer may have a hinge-type characteristic or function. The spacer may also allow limit the steric hindrance of the support, thereby allowing the biologically active entity to perform its intended biological reaction.

The spacer may also be linked to a first linker, such as a polyaldehyde compound. As used herein, the term “polyaldehyde” is intended to mean a compound having more than one aldehyde group. An aldehyde is either a functional group consisting of a terminal carbonyl group or a compound containing a terminal carbonyl group. In organic chemistry, functional groups are specific groups of atoms within molecules, that are responsible for the characteristic chemical reactions of those molecules. The same functional group will undergo the same or similar chemical reaction(s) regardless of the size of the molecule it is a part of. The first linker should therefore be made of at least two aldehyde groups and is used as a chemical bridge between the spacer and the biologically active entity. This chemical bridge binds covalently the spacer and the biologically active entity. In an embodiment, the first aldehyde group of the first linker is linked to the spacer, while the second aldehyde group of the first spacer is linked to the biologically active entity. In an embodiment, the first and second aldehyde group of the first linker may be derived from polymers such as, but not limited to, glutaraldehyde, glutardialdehyde, 1,3-diformylpropane, glutaral, 1,5-pentanedial, 1,5-pentanedione and cidex. Other polyaldehydes can be used to perform the system and method of the present invention. In an embodiment, the first linker is glutaraldehyde.

In a further embodiment, a second linker may also be added to the system. This second linker may also a be polyaldehyde compound. The second linker should therefore be made of at least two aldehyde groups and is used as a chemical bridge between the surface of the support and the spacer. This chemical bridge binds covalently the surface of the support to the spacer. In an embodiment, the first aldehyde group of the second linker is linked to the polyaldehyde compound of the first spacer while the second aldehyde group of the second spacer is linked to the support. The aldehyde groups of the second spacer may be derived from glutaraldehyde, glutardialdehyde, 1,3-diformylpropane, glutaral, 1,5-pentanedial, 1,5-pentanedione orcidex. In an embodiment, the second linker is glutaraldehyde.

The biologically active entity present in the system is indirectly linked to the spacer. In an embodiment, the biologically active entity is directly linked to the first linker. On the other hand, the first linker can allow covalent bonding between the biologically active entity and the support. Without wishing to be bound to theory, the first linker may link through one of its aldehyde group to the biologically active entity.

The biologically active entity may include any molecule, cell or cell portion, organelle or organelle portion, protein or protein function that is capable of having a biological or biochemical activity, such as for example, but without limitation, an enzyme, a co-enzyme, an antibody, a prokaryotic or eukaryotic cell, a cell portion, a yeast, a bacteria, or a fungus product.

In an embodiment, the enzyme may, for example, but without limitation, include a hydrolase, an anhydrase, a protease, or any other enzyme of interest. In an embodiment, the anhydrase is a carbonic anhydrase (such as a human, a mammal or a plant anhydrase). An antibody or binding fragment thereof can also be fixed to a support according to the system and method presented herein, and be used, for example but not limited to, in a purification column or device as it is well known in the art and laboratories.

The system described herein enables the processing of a biological reaction by a biologically active entity immobilized on a support by means of a spacer and a first linker (and optionally second spacer). This system provides an increased activity and a higher stability of the immobilized biologically active entity.

As used herein, the term “biological reaction” includes, among others, enzymatic reactions. The enzyme, for example, may be fixed on a support according to the system and method described herein and can be placed in a bioreactor to perform biological reaction. Additional reagents or substrates can be placed in the bioreactor. The same can be applied on a system into with fixed microorganisms or eukaryotic cells. The biologically active entities placed in this configuration provides a bioactive surface capable of catalyzing a biological reaction.

In an embodiment, the biological reaction is the conversion of toxic gaseous effluents in less toxic or non-toxic by-products. Gaseous effluents include, but are not limited to greenhouse gases such as CO₂, CO and methane. As used herein the expression “less toxic or non-toxic by-products” is intended to mean a by-product produced by the biolological reaction having a cytotoxicity less than the toxic gaseous effluent. Examples of such biological reactions include, but are not limited to the conversion of CO₂ by carbonic anhydrase, the conversion of CO₂ by rubisco, the conversion of CO by monooxydases, and the conversion of formaldehydes by oxygenases ore deshydrogenases.

In another embodiment, the biological reaction can take place into an aqueous solution. In this particular embodiment, the gaseous effluent is dissolved in the aqueous solution in order to put it in contact the biologically active entity.

It will also be understood by the man of the art that the system described herein could be used for various applications such as, for example, air purification , treatment of gaseous effluents, etc.

All the possible forms of carbonic anhydrase may be considered in the system described herein (and methods relating thereto). In particular, carbonic anhydrase may be of natural origin, i.e. purified directly from a source, or obtained by recombinant methods known in the art. A functional moiety of this enzyme having the catalytic activity of the native enzyme may also be used for carrying out the present invention.

According to one of the embodiment of the present invention, the system described herein makes use of carbonic anhydrase (CA. EC 4.2.1.1.) which is a metallo-enzyme containing zinc, and which is capable of catalyzing reversible hydration of carbon dioxide. The reaction carried out by the enzyme is the following:
CO₂+H₂O⇄H⁺+HCO₃³¹

Its catalytic activity significantly accelerates the natural reaction of conversion of dissolved carbon dioxide into bicarbonate ions. These ions may thereafter be converted into precipitate by contact with bivalent ions, such as for example, but without limitation, of the calcium or magnesium type, thus revaluating industrial wastes.

It should be understood by one skilled in the art that the system comprising a carbonic anhydrase enzyme immobilized on a support could also be found in different environments, such as for example in a system for the purification or recirculation of air or in a bioreactor through which the source containing carbon dioxide will be able to circulate and be treated or converted.

Sequestration of carbon dioxide in the form of a chemically stable carbonate product is a phenomenon that is made possible by means of the present system and method in the long-lasting development of a technology aiming at reducing greenhouse effect gases, or at converting and reducing carbon dioxide in different environments.

According to one embodiment of the present invention, as a factor which activates the carbon dioxide hydration reaction, carbonic anhydrase is bonded to a support, preferably a solid support. Carbon dioxide must be dissolved to achieve its catalysis through the enzyme, the support-enzyme complex, while in this configuration, having the advantage of a wettability, which facilitates transport of carbon dioxide dissolved at the active site of the enzyme. The term “wettability” as used here means a higher hydrophilicity that can be obtained by increasing the functional sites on the support or still through a better adapted ‘spacer’ molecule, i.e. more polar. A better hydrophilicity means the possibility for a molecule to produce hydrogen bridges with a molecule of water at the surface of the support.

Therefore, in another embodiment, the present invention provides a method for obtaining an immobilized biologically active entity. The method may comprise the step of providing a support linked to a spacer and a first linker as described herein. The method may also comprise linking the biologically active entity to the first linker. The method may also comprise providing a support linked to a second linker, a spacer and a first linker. The support may also be functionalized prior to its use in the method (refer to the functionalization steps described above). The functionalization of the support may include, but is not limited to, the creation of primary amine groups on the surface of the support or by the addition of primary amine groups on the surface of the support. The method can also comprise the addition of amino acids to the support in order to block the charged zones of its surface.

The advantages of the system and method described herein are numerous. In the preparation of the system, washings are mostly carried out with dechlorinated water from the waterworks system which contributes to minimize production costs. In addition, a reduction in the number of steps lowers the involvement of chemical molecules (potentially harmful) in the process of binding the biologically active entity. Further, the use of similar buffer rather than many different buffers for each of the chemical products reduces the number of chemical compounds. Finally, the method described herein generate a stable immobilization in a very short time lag.

The system (and method relating thereto) described herein enables the biologically active entity to retain a good biological activity stability and increases the yield obtained with respect to methods and systems disclosed in the literature. The system (and method relating thereto) is cost-efficient since it enables the immobilization of the biologically active entity in a shorter period (e.g. in less than four, less than two or less than one day) with fewer steps. In addition, the system presented herein permit sustainable development; by reducing the carbon life cycle.

Although the present invention has been described through some more specific embodiments, it will be agreed that other modifications may be applied to the present invention and that these applications are intended to cover different variants, uses, or adaptations of the invention following in a general manner its basic principle as will be understood by the man of the art.

The present invention will be more readily understood by referring to the following examples which are given to illustrate the invention rather than to limit its scope.

### EXAMPLE I

### Carbonic Anhydrase System Immobilized With Two Linkers On A Solid Support

Immobilization.

Methanolysis of the solid support with a Raschig ring geometry (GE polymer shapes) was performed at 50° C. during 60 minutes. The remaining steps have been performed at room temperature. The support was then washed 5 times with dechlorinated water. Hydrolysis of the support was performed with an HCl colution (3.93 N, obtained from Lab Mat) for 1 hour. The support was then washed 5 times with dechlorinated water and with NaOH (0.1M, obtained from Lab Mat) for 1 hour. The support was then washed 12 times with dechlorinated water. The support was pretreated by immersion in a carbonate buffer (Sigma, 0.2M pH 8.5) for 1 hour. The support was then treated with a glutaraldehyde (Sigma) solution (2.5% in a carbonate buffer 0.2M pH 8.5) for 1 hour. The support was then washed 5 times with dechlorinated water. The support was incubated 18 hours in a polyethylenimine (PEI, obtained from Sigma) solution (0.5% in a phosphate buffer 0.1M pH 8.0). The support was then washed 5 times with dechlorinated water. The support was then blocked with a mixture of amino acids (L-phenylalanine, D-leucine, L-arginine, glycine, D- and L-aspartic acid, obtained from Sigma) solution (0.5% in a phosphate buffer solution 0.1M pH 8.0). The support was then washed 5 times with dechlorinated water. The support was pretreated with a carbonate buffer 0.2 M pH 8.5 for 1 hour. The support was treated with a glutaraldehyde 2.5% solution in a carbonate buffer 0.2 M pH 8.5 for 15 minutes. The support was then washed 5 times with dechlorinated water. The enzyme (carbonic anhydrase isolated from human blood and obtained from CO₂ Solution) was then added to the support at a concentration of 1.0 mg/ml in a carbonate buffer for 2 hours. The support was then washed 4 times with dechlorinated water, 1 time with a NaCl (Sigma) solution (1.0 M) and 4 times with dechlorinated water. The immobilization was completed in a period of four (4) days.

This method allows for the covalent immobilization of carbonic anhydrase on a support having hydrophilic character, the enzyme being held through covalent bonds to the support. This method also provides enzyme activity and stability superior to what is currently known in the art.

Assessment of CO₂ conversion.

The ability of the immobilized enzymes to convert CO₂ into carbonate has then been tested using the following conditions. Raschig™rings were placed at random in a column (50 cm high and 8 cm diameter). The liquid flow ( Tris 12 mM solution) was adjusted to 0.5 L/min. The gas flow (pure CO₂ 10 000 ppm) was adjusted to 1.0 g/min. The yield of CO₂ conversion was calculated using the following formula:
[Quantity of CO₂ input(ppm)−Quantity of CO₂ output(ppm)/Quantity of CO₂ input(ppm)]×100.

The results obtained with an untreated support having no immobilized enzyme (Raschig Ring™ crude) were compared to those of the supports prepared by the method described above (Raschig Ring™ ACHIIr). The results obtained are presented in Table 1.

TABLE 1
Conversion yield in function of type of support used

| | Yield (%) |
|---|---|
| Raschig Ring ™ Crude | 67 +/− 4 |
| Raschig Ring ™ ACHIIr | 84 +/− 3 |

The results presented herein show that the use of a spacer for the immobilization of CO₂ converting enzyme increases the stability of the enzyme when compared to what is presently known in the art, and preserves a good level of activity of carbon dioxide conversion. Moreover, the system described herein permits the use of different supports and shapes, made from a variety of materials.

### EXAMPLE II

### Carbonic Anhydrase System Immobilized With A Single Linker On A Solid Support

Immobilization.

Every step of this immobilization method was performed at room temperature. The chemicals/biologicals used are the same as those described in Example I. The enzyme carbonic anhydrase was immobilized on Raschig™ rings (5kg) made of Nylon 6/6. The solid support was hydrolyzed for 1 hour with a HCl solution (3.93 N). The support was then washed 6 times with dechlorinated water and 1 time with a NaOH solution (0.1 M). The support was further washed 4 times with dechlorinated water. The support was then incubated between 2 and 18 hours with a PEI solution (concentration of 0.5M in a carbonate buffer 0.2 M pH 8.3). The length of the incubation was adjusted to the physico-chemical conditions sought. The support was then left to drain and was not washed prior to its incubation with glutaraldehyde. The support was incubated 2 hours in a glutaraldehyde solution (1.0% in carbonate buffer 0.2 M pH 8.3). The support was then incubated 2 hours in a carbonhic anhydrase solution (0.5 mg/ml). The support was then washed 3 times with dechlorinated water and 1 time with a NaCl solution (1.0M). The support was finally washed 3 times with dechlorinated water. This procedure can be perfomed in a single day or it may be divided into two days at the step of adding the polyethylenimine to facilitate working hours. In the latter, the solid support may then be placed in contact with the polyethylenimine during the entire night. The use of a single step of glutaraldehyde addition contributes to not only reducing the production time and its cost but also the reduction of production of toxic waste.

Assessment of CO₂ Conversion.

The ability of the carbonic anhydrase immobilized on solid supports to convert CO₂ into carbonate has then been verified. The support was distributed at random in a column 50 cm high by 8 cm diameter. The liquid flow of a Tris solution (12 mM) was adjusted to 0.5 L/min. The gaseous flow (CO₂ 10 000 ppm ) was adjusted to 1.0 g/min The ability of the immobilized carbonic anhydrase to convert CO₂ was then assessed. The ability of the immobilized carbonic anydrase described in Example I was compared to the immobilized in the present Example.

TABLE 2
Hydratation of CO₂ in a lined column

| | Yield increase with |
|---|---|
| | respect to the crude |
| | support as described |
| | in Example I |
| Immobilized enzyme described in Example I | 82.4 +/− 2.5%. |
| Immobilized enzyme described in Example II | 97.4 +/− 2.5%. |

In conclusion, the method and the system of the present invention makes it possible to obtain the immobilization of biologically active entities having an improved stability as compared to what is known in the art, and a good level of activity, especially in the conversion of carbon dioxide. Moreover, the system permits the use of various supports, of very different shapes and made of different materials.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A system for performing a biological reaction, said system consisting essentially of:
a support;
a first spacer having a polyamine molecule;
a first linker having a having a first aldehyde group and a second aldehyde group; and
a biologically active entity;
wherein said support is linked to the polyamine molecule of said spacer, wherein said spacer is linked to the first aldehyde group of said first linker and wherein said biologically active entity is linked to the second aldehyde group said first linker.

2. The system of claim 1, further containing a second linker having a first aldehyde group and a second aldehyde group, wherein the first aldehyde group of said second linker is linked to the polyamine molecule of said spacer and the second aldehyde group of said second linker is linked to said support.

3. The system of claim 1, wherein said support is made of a compound selected from the group consisting of plastic, biopolymer, polytetrafluoroethylene (PTFE), ceramic, polyethylene, polypropylene, polystyrene, nylon, silica, carbonate, a derivative thereof and a combination thereof.

5. The system of claim 1, wherein the polyamine molecule of said spacer is selected from the group consisting of an hydrocarbon, an acyclic hydrocarbon an alkene, a polyene, a polyethylene, an imine and a polyethylenimine.

6. The system according to claim 1, wherein the polyamine molecule of said spacer is hydrophilic.

7. The system according to claim 1, wherein the polyamine molecule of said spacer is polyethylenimine.

8. The system of claim 1, wherein the first linker is selected from the group consisting of glutaraldehyde, glutardialdehyde, 1 ,3-diformylpropane, glutaral, 1,5-pentanedial, 1,5-pentanedione and cidex.

9. The system according to claim 1, wherein said first linker is glutaraldehyde.

10. The system of claim 2, wherein the second linker is selected from the group consisting of glutaraldehyde, glutardialdehyde, 1,3-diformylpropane, glutaral, 1,5-pentanedial, 1,5-pentanedione and cidex.

11. The system according to claim 2, wherein the second linker is glutaraldehyde.

12. The system of claim 1, wherein said biologically active molecule is an enzyme.

13. The system of claim 12, wherein said enzyme is carbonic anydrase.

14. The system of claim 1, wherein said biological reaction is the conversion of a toxic gaseous effluent into a lesser toxic by-product.

15. The system of claim 1, wherein said biological reaction is the conversion of carbon dioxide into carbonate.

16. The system of claim 1, wherein said biological reaction takes place in an aqueous solution.

17. A method for obtaining an immobilized biologically active entity, said method consisting essentially of the steps of:
a) providing a support linked to a spacer, said spacer having a polyamine molecule, said spacer being linked to a first linker, said first linker having a first aldehyde group and a second aldehyde group, wherein the first aldehyde group of the first linker is linked to said spacer; and
b) linking a biologically active entity to the second aldehyde group of said first linker; thereby obtaining said immobilized biologically active entity.

18. The method of claim 17, wherein said support is linked to a second linker, said second linker having a first aldehyde group and a second aldehyde group, wherein the first aldehyde group of said second linker is linked to said support and the second aldehyde group of said second linker is linked to said spacer.

19. The method of claim 17, wherein said support is made of a compound selected from the group consisting of plastic, biopolymer, polytetrafluoroethylene (PTFE), ceramic, polyethylene, polypropylene, polystyrene, nylon, silica, carbonate, a derivative thereof and a combination thereof.

20. The method of claim 17, wherein said support is hydrolyzed by acid or functionalized by an ammoniacal plasma treatment prior to step a).

21. The method of claim 20, wherein acid hydrolysis causes the linking of the polyamine molecule of said spacer to said support.

22. The method of claim 20, wherein acid hydrolysis creates primary amine groups on said support.

23. The method of claim 22, wherein said primary amine group is selected from the group consisting of aminobutane, 2-amino-2-methylpropane, 1-methylaminopropane, dimethylpropane, sulfonamide, alkoxide, amide and Barton's base.

24. The method of claim 17, wherein the polyamine molecule of said spacer is selected from the group consisting of an hydrocarbon, an acyclic hydrocarbon an alkene, a polyene, a polyethylene, an imine and a polyethylenimine.

25. The method of claim 17, wherein the polyamine molecule of said spacer is hydrophilic.

26. The method of claim 17, wherein the polyamine molecule of said spacer is polyethylenimine.

27. The method of claim 17, wherein said first linker is selected from the group consisting of glutaraldehyde, glutardialdehyde, 1,3-diformylpropane, glutaral, 1,5-pentanedial, 1,5-pentanedione and cidex.

28. The method of claim 17, wherein said first linker is glutaraldehyde.

29. The method of claim 18, wherein said second linker is selected from the group consisting of glutaraldehyde, glutardialdehyde, 1,3-diformylpropane, glutaral, 1,5-pentanedial, 1,5-pentanedione and cidex.

30. The method of claim 18, wherein said second linker is glutaraldehyde.

31. The method of claim 17, wherein said biologically active molecule is an enzyme.

32. The method of claim 31, wherein said enzyme is carbonic anydrase.

33. The method of claim 17, wherein the steps are performed at room temperature.
